(19) United States
(12) Patent Application Publication
(10) Pub. No.: US 2026/0317715 A1
(43) Pub. Date: Oct. 15, 2026

(54) METHOD FOR PRODUCING ERYTHROCYTES FROM CANINE HEMATOPOIETIC STEM CELLS OR ERYTHROID PROGENITOR CELLS THROUGH IN VITRO CULTURE

(71) Applicant: IUCF-HYU (INDUSTRY-UNIVERSITY COOPERATION FOUNDATION HANYANG UNIVERSITY), Seoul (KR)

(72) Inventors: Eun Jung BAEK, Seoul (KR); Sung Ah PARK, Seoul (KR)

(21) Appl. No.: 19/685,047

(22) Filed: May 21, 2026

Related U.S. Application Data
(63) Continuation of application No. PCT/KR2024/019009, filed on Nov. 27, 2024.

(30) Foreign Application Priority Data
Nov. 27, 2023 (KR) 10-2023-0166331

(51) Int. Cl. C12N 5/078

(52) U.S. Cl. CPC C12N 5/0641; C12N 2500/25; C12N 2500/32; C12N 2501/125; C12N 2501/14; C12N 2501/2303; C12N 2501/33; C12N 2506/11

(57) ABSTRACT

Provided herein are a method for differentiating canine hematopoietic stem cells into erythrocytes through in vitro culture, and a method for producing canine erythrocytes using the in vitro culture. In accordance with the method, erythrocytes can be effectively produced from canine hematopoietic stem cells by using cytokines at specific concentrations.

### TECHNICAL FIELD

The present invention relates to a method for culturing canine hematopoietic stem cells or erythroid progenitor cells in vitro to differentiate and proliferate them into erythrocytes, and a method for producing canine erythrocytes using the same.

### BACKGROUND ART

Blood transfusions in dogs play a crucial role in veterinary medicine, both in managing anemia and managing acute blood loss due to trauma or surgery. Blood donation is the sole source of blood transfusions. Despite the widespread availability of advanced veterinary treatments, the demand for blood products is increasing. However, the supply of blood from companion animals is limited, and with ethical issues surrounding blood donation animals being raised, the need for extracorporeally produced erythrocytes is increasing.

In the case of humans, research into in vitro production has been conducted for many years due to a shortage of blood donations, and clinical trials are currently underway. However, even basic methods for producing erythrocytes in dogs have not been studied, the related reagents are not readily available, and it is also unclear whether the culture medium is appropriate. A study by Leberbauer et al. (2005) showed that erythrocytes derived from cord blood mononuclear cells (MNCs) can be expanded up to 1×10⁹ times. The researchers successfully produced red blood cells (RBCs) that matured simultaneously and had a very high enucleation rate, making them indistinguishable from red blood cells in peripheral blood (Reference 1). In 2011, a French research team, Douay et al., reported the first case of transfusing 2 mL of blood cultured with CD34+ cells obtained from an autologous blood sample into a human recipient (Reference 2). Recently in the UK, a clinical trial of blood transfusion using laboratory-cultured red blood cells (Recovery and survival of stem cell originated red cells: RESTORE) was attempted.

In contrast, studies on erythropoiesis, cell proliferation, differentiation, and hematopoiesis using mononuclear cells in dogs are relatively limited. H.-J. Kolb et al. investigated the immunophenotype of canine hematopoietic progenitor cells and explored potential therapeutic applications and disease models (Reference 3).

In most cases, these studies aimed to understand immune responses and cellular functions in various situations by culturing immune cells in vitro. S-K KIM et al. demonstrated that canine NK cell subtypes exhibited phenotypic transformation and described NK cell biology and potential therapeutic applications through in vitro mononuclear cell culture (Reference 4).

Nevertheless, no research has yet been conducted on culturing erythrocytes derived from mononuclear cells in vitro. Canine erythrocytes maintain a smooth and concave surface similar to human erythrocytes, and their average diameter increases slightly with age (Reference 5). Cross-species metabolomic analysis of erythrocytes has revealed that canine erythrocytes exhibit stronger antioxidant capacity and energy metabolic characteristics (Reference 6). However, currently there is a significant lack of data and research results regarding in vitro erythrocyte studies.

### DISCLOSURE

### Technical Problem

Under the circumstances described above, the present inventors studied a method capable of producing canine erythrocytes and confirmed that by adjusting the concentration of canine EPO, canine mononuclear cells can be effectively differentiated into erythrocytes, thereby completing the present invention.

Therefore, an object of the present invention is to provide a method for producing erythrocytes by differentiating and proliferating canine hematopoietic stem cells or erythroid progenitor cells into erythrocytes.

Another object of the present invention is to provide erythrocytes produced by the method.

### Technical Solution

To achieve the above objects, one aspect of the present invention provides a method for producing canine erythrocytes in vitro, the method including:
obtaining hematopoietic stem cells or erythroid progenitor cells from canine-derived cells; and
culturing the obtained hematopoietic stem cells or erythroid progenitor cells in a medium containing erythropoietin (EPO).

In the present invention, the canine-derived cells may be blood-derived cells, bone marrow-derived cells, cord blood-derived cells, or stem cells. Further, the stem cells may be embryonic stem cells (ESCs), induced pluripotent stem cells (iPS cells, iPSCs), but are not limited thereto.

A method for isolating hematopoietic stem cells or erythroid progenitor cells from the canine-derived cells may include a method known in the art to which the present invention pertains. For example, when the canine-derived cells are blood-derived cells, hematopoietic stem cells may be isolated using Ficoll, and the like, and when the canine-derived cells are stem cells, hematopoietic stem cells or erythroid progenitor cells may be isolated by culturing the stem cells in a medium that induces differentiation into hematopoietic stem cells/erythroid progenitor cells.

The term erythroid precursor cells used in the present invention refers to all cells involved in the erythrocyte formation process except for mature erythrocytes, and refers to erythroid cells before enucleation. For example, the erythroid precursor cells may be CD34+ cells. In addition, the erythroid precursor cells may be selected from the group consisting of a burst-forming unit-erythroid (BFU-E), a colony-forming unit-erythroid (CFU-E), a proerythroblast, a basophilic erythroblast, a polychromatic erythroblast, an orthochromatic erythroblast, and a reticulocyte.

The term hematopoietic stem cells used in the present invention refers to cells that account for approximately 0.1% of the blood of a healthy person and have the ability to produce all blood cells. Hematopoietic stem cells are found throughout the body and are mass-produced in the bone marrow. In the present invention, hematopoietic stem cells refer to cells capable of producing blood lineage cells through differentiation or other methods.

According to an embodiment of the present invention, the hematopoietic stem cells may be mononuclear cells (MNCs). Mononuclear cells are peripheral blood cells with a round nucleus and consist of lymphocytes (T cells, B cells, NK cells) and monocytes.

In the present invention, the erythropoietin may be included in the medium at a concentration of 20 to 80 ng/ml, and preferably may be included in the medium at a concentration of 30 to 50 ng/ml.

The present inventors confirmed the effect of the concentration of erythropoietin on the process of differentiating canine mononuclear cells into erythrocytes, and confirmed that increasing the concentration of erythropoietin resulted in differences in cell proliferation rate, enucleation, erythrocyte maturation, and lineage differentiation (FIG. 5). In particular, increasing the concentration of EPO did not increase cell proliferation and erythrocyte differentiation efficiency, and the present inventors confirmed that cell proliferation and erythrocyte differentiation efficiency were the best when canine recombinant EPO was used at a concentration of 40 ng/ml (FIGS. 5 and 7).

Accordingly, in the present invention, the erythropoietin may be canine erythropoietin, and may be added to the medium at a concentration of 5 to 80 ng/ml, preferably 20 to 60 ng/ml, and most preferably 40 ng/ml.

The present inventors also confirmed the effect of human erythropoietin and confirmed that erythrocytes can be induced from canine mononuclear cells even though the efficiency is lower than that of canine erythropoietin. Therefore, the erythropoietin may be human erythropoietin.

In the present invention, the medium may further contain transferrin, insulin, penicillin/streptomycin, and amino acids in addition to cytokines.

Furthermore, the medium may further contain stem cell factor (SCF) and interleukin-3 (IL3) as cytokines in addition to erythropoietin. Stem cell factor may be included in the medium at a concentration of 10 to 100 ng/ml, and interleukin-3 may be included in the medium at a concentration of 1 to 10 ng/ml.

The present inventors confirmed that when canine mononuclear cells were cultured in a medium containing erythropoietin, the number of cells temporarily decreased in the early stage of culture, but erythrocytes without nuclei were observed from day 10 of culture (FIG. 2), and the number of cells reached its maximum on day 17 of culture (FIG. 5). Therefore, in the present invention, the culturing step may be performed for 3 days to 30 days such that the cells sufficiently proliferate beyond the period of cell number decrease. Preferably, the culturing step may be performed for 7 days to 25 days.

Another aspect of the present invention provides erythrocytes produced according to the method.

The present inventors measured the oxygen equilibrium curves of erythrocytes produced by the method of the present invention and erythrocytes isolated from dogs, and confirmed that the oxygen-Hb binding and dissociation curves were similar in the two types of erythrocytes (FIG. 13B). These results indicate that erythrocytes produced by the method of the present invention can produce hemoglobin, and can be used for blood transfusion in canines.

### Advantageous Effects

According to the method of the present invention, by controlling the concentration of canine EPO, canine hematopoietic stem cells can be effectively differentiated into erythrocytes, and canine erythrocytes can be produced.

### DESCRIPTION OF DRAWINGS

FIG. 1 schematically illustrates a method for differentiating canine mononuclear cells into erythrocytes according to one embodiment of the present invention.

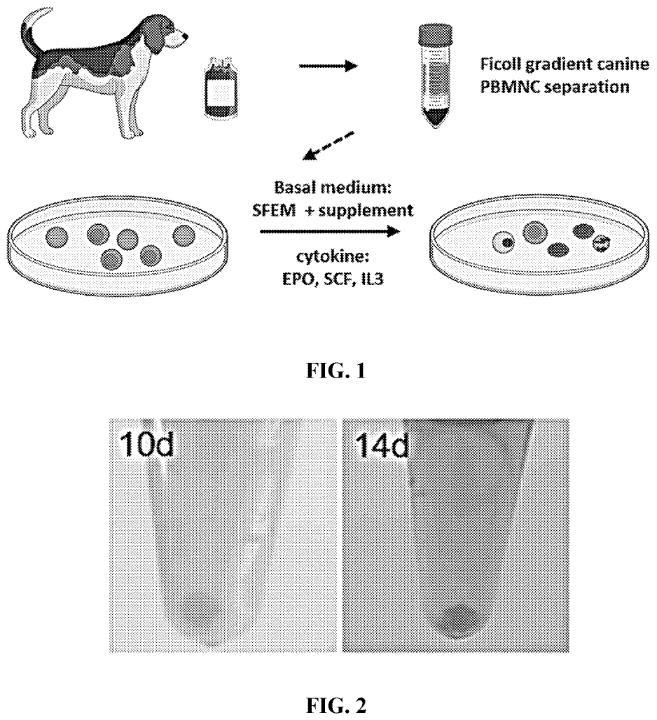

FIG. 2 shows the results of confirming a cell pellet by harvesting cells on day 10 and day 14 of culture in the process of differentiating canine mononuclear cells into erythrocytes.

FIG. 3 shows the results of observing cell morphological changes in the process of differentiating canine mononuclear cells into erythrocytes (crEPO40 ng/ml): red arrows—red blood cells (RBCs); and yellow arrows—orthochromatic erythroblasts.

FIG. 4 schematically illustrates the morphological changes of cells in the erythropoiesis process in humans and dogs.

FIG. 5 is a graph confirming the number of cells proliferating while differentiating canine mononuclear cells into erythrocytes using media containing different concentrations of crEPO.

FIG. 6 is a graph confirming cell viability while differentiating canine mononuclear cells into erythrocytes using media containing different concentrations of crEPO.

FIG. 7 is a graph confirming the number of erythroid cells on day 17 of culture while differentiating canine mononuclear cells into erythrocytes using media containing different concentrations of crEPO: RBC—red blood cells; ortho—orthochromatic erythroblasts; pro-baso-poly—proerythroblasts, basophilic erythroblasts, polychromatic erythroblasts; and others—other cells.

FIG. 8 is a graph confirming the percentage of erythroid cells according to culture day while differentiating canine mononuclear cells into erythrocytes using a medium containing crEPO 40 ng/ml: RBC—red blood cells; ortho—orthochromatic erythroblasts; pro-baso-poly—proerythroblasts, basophilic erythroblasts, polychromatic erythroblasts; and others—other cells.

FIG. 9 is a graph confirming the number of cells proliferating while differentiating canine mononuclear cells into erythrocytes using media containing different concentrations of crEPO or human EPO.

FIG. 10 is a graph confirming cell viability while differentiating canine mononuclear cells into erythrocytes using media containing different concentrations of crEPO or human EPO.

FIG. 11 is a graph confirming the number of erythroid cells on day 17 of culture while differentiating canine mononuclear cells into erythrocytes using media containing different concentrations of crEPO or human EPO: RBC—red blood cells; ortho—orthochromatic erythroblasts; pro-baso-poly—proerythroblasts, basophilic erythroblasts, polychromatic erythroblasts; and others—other cells.

FIGS. 12A, 12B, 12C and 12D show the results of analyzing the expression of erythroid markers on days 0, 10 to 13, and 15 to 17 of culture while differentiating canine mononuclear cells into erythrocytes using a medium containing crEPO 40 ng/ml or 80 ng/ml: a—alpha-hemoglobin stabilizing protein (AHSP); b—EPO receptor (EPOR); c—Kruppel-like factor (KLF1); and d—transferrin receptor 1 (TFRC).

FIGS. 13A, 13B and 13C show the results of confirming the function of erythrocytes produced from canine mononuclear cells through in vitro culture: a—appearance of cell pellet on day 18 of culture; b—oxygen-Hb binding and dissociation curves of canine PBMCs cultured in vitro for 18 days and canine erythrocytes on day 1; c—results of confirming the expression of hemoglobin α in canine PBMCs cultured in vitro for 17 days.

FIG. 14 shows the results of comparing the amino acid sequences of human EPO and canine EPO.

FIG. 15 shows the results of comparing the amino acid sequences of human EPOR and canine EPOR.

### MODES OF THE INVENTION

Hereinafter, one or more specific exemplary embodiments will be described in more detail through examples. However, these examples are provided only for exemplarily describing the one or more specific exemplary embodiments, and the scope of the present invention is not limited to these examples.

### Experimental method

### Isolation of Mononuclear Cells (MNCs) From Canine Peripheral Blood

The canine peripheral blood used in this study was approved by the Institutional Animal Care and Use Committee (IACUC) of Hanyang University. Whole blood and Ficoll were layered in a 1:1 ratio using Ficoll-Paque (1.078 g/ml, Cytiva, #17144003) in SepMate (SepMate™ PBMC Isolation Tubes, STEMCELL Technologies) and centrifuged at 500×g for 35 minutes at room temperature. After identifying the plasma supernatant and mononuclear cell layer, only the mononuclear cell layer was collected. The collected mononuclear cell layer was diluted in PBS containing 10% FBS and washed by centrifugation at 800×g for 20 minutes at room temperature. The isolated cells were collected to measure the number of mononuclear cells.

### Cell Culture

Peripheral blood mononuclear cells (PBMCs) isolated from dogs were cultured in StemSpan™ (STEMCELL Technologies) medium containing 30% FBS (HyClone™) and basic supplements. The basic supplements include Holo-Transferrin (200 ng/ml, Sigma-Aldrich, T4132), insulin (1 μl/ml), 1% penicillin/streptomycin, and 1% amino acids. The cytokines used were canine recombinant EPO (3816-CE 010/C, R&D System), canine recombinant SCF (10 to 100 ng/ml, 2278-SC 025/CF, R&D Systems), and human recombinant IL3 (human rIL3: 1 to 10 ng/ml, 203-IL-050, R&D Systems), and cytokine conditions varied depending on the specific experiment. 90% of the cell culture medium was replaced with fresh medium every 2 to 3 days.

### Analysis of Cell Morphology Using Wright-Giemsa Staining

Cultured erythroid cells (2 to 4×10⁴ cells) were smeared on slides using a cell centrifuge (Cellspin; Hanil Science Industrial, Incheon, Korea) and stained with Wright-Giemsa (Sigma), and then cell morphology was observed.

### Western Blot

The expression level of canine hemoglobin α was analyzed by Western blot. The cultured cells were collected, washed with PBS, and then lysed with radioimmunoprecipitation assay (RIPA, Sigma) buffer. The isolated proteins were quantitatively analyzed with the Pierce BCA protein kit (Thermo Scientific). A certain amount of protein was electrophoresed on a 10% SDS-PAGE gel and then transferred to a PVDF membrane (Millipore Corporation, MA). The membrane containing the transferred protein was blocked with 5% skim milk and then incubated with a primary antibody (Anti-Hemoglobin antibody, ab231732) and a secondary antibody. Protein detection was performed using the ChemiDoc XRS+ system (Bio-Rad, Hercules, CA).

### Functional Analysis of Canine Erythrocytes Cultured In Vitro

On day 18 of culture, the oxygen affinity of hemoglobin was determined using canine erythrocytes. The oxygen equilibrium curve was measured using a Hemox-Analyzer (TCS, Medical Products Division, Southampton, PA).

### Experimental Results

### Erythropoiesis Characteristics Differentiated From Canine PBMCs

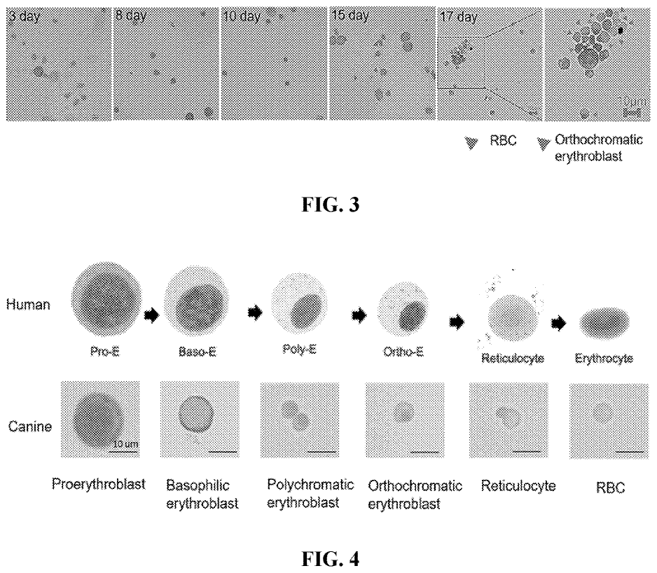

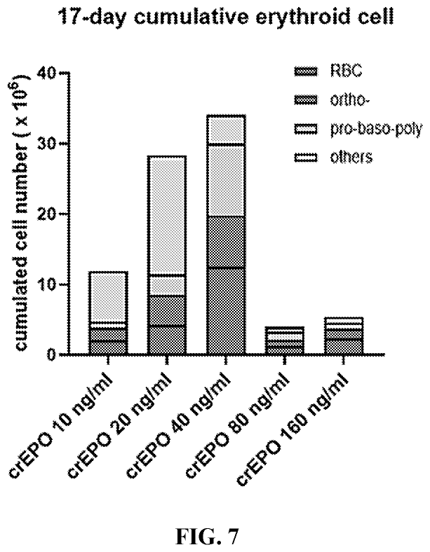

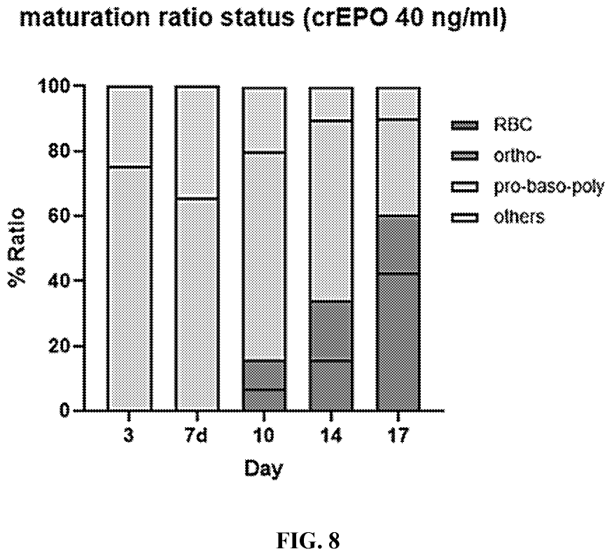

After isolating canine PBMCs, they were seeded at a density of 1×10⁶ cells/ml, and their proliferation and differentiation were observed for 3 weeks. Platelets were present in the PBMCs for the first 6 to 7 days, but gradually disappeared. From day 0 to day 6, the proportion of seeded cells decreased to 20% (FIG. 5). However, proliferation began thereafter, and from day 10, erythrocytes without nuclei were observed, and the cells began to turn red (FIG. 2). The period of maximum proliferation compared to the initially seeded cells appeared on days 16 to 17 of proliferation, and there were differences in the proliferation rate depending on the donor. Cell viability was also generally maintained at a high level (FIG. 6).

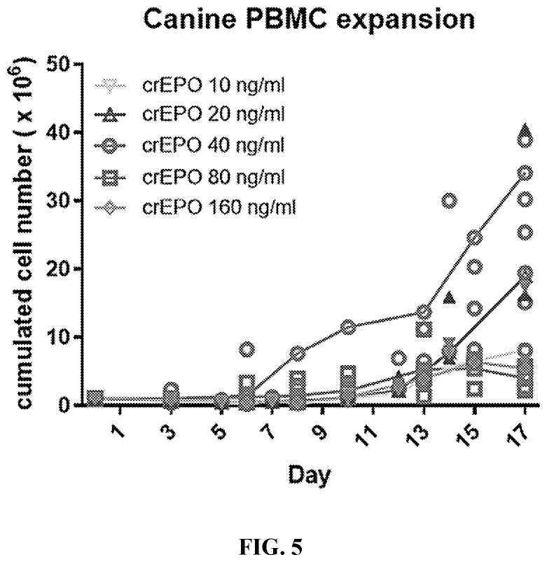

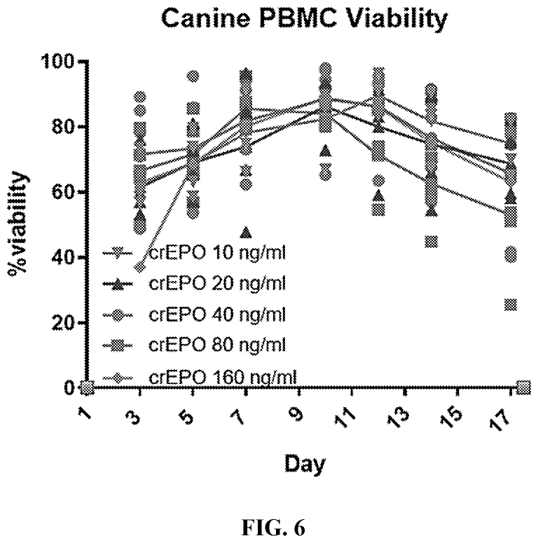

The membranes of mature differentiated erythrocytes were maintained intact, and the differentiation stage of the cells could be confirmed by Wright-Giemsa staining (FIGS. 3 and 4).

### Optimal Cytokine Concentration

To determine the optimal conditions for proliferation and differentiation of erythroid lineages, the concentration of canine recombinant EPO (crEPO) was investigated. The concentrations of crEPO were set to 10, 20, 40, 80, and 160 ng/ml, and cultured for about 20 days.

Regardless of cytokine concentration, the number of cells decreased until day 7 after inoculation, and thereafter, the total number of cells increased as cells began to proliferate. Although a concentration of 40 ng/ml of crEPO was used as a reference, there was no significant difference in cell morphology and proliferation rate at any concentration of crEPO for the first 7 days.

From day 13 of culture, cell aggregation was observed at low concentrations of 10 ng/ml and 20 ng/ml, and cells began to form island-like structures (data not shown). From days 14 to 15 of culture, differences in enucleation, erythroid maturation, and lineage differentiation began to appear. After day 15 of culture, the cell proliferation rate of the 20 ng/ml crEPO group decreased, and the cell number of the 40 ng/ml crEPO group increased 1.35-fold (FIG. 5). In addition, the cell number of the 10 ng/ml crEPO group was 3.18-fold lower than that of the 40 ng/ml crEPO group and 2.34-fold lower than that of the 20 ng/ml crEPO group.

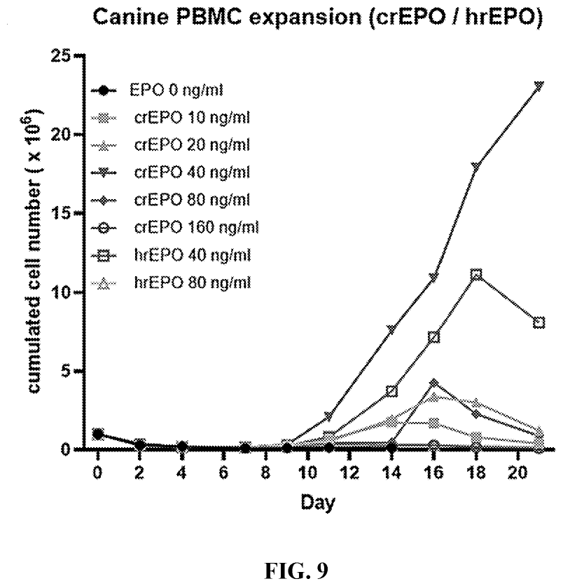

On day 17 of culture, the proportions of erythrocytes were shown to be 36.8% (n=7) in the 40 ng/ml crEPO group, 15.12% (n=2) in the 20 ng/ml crEPO group, and 17.94% (n=2) in the 10 ng/ml crEPO group. The total erythrocyte number and cell number were highest in the 40 ng/ml crEPO group. Compared to the 40 ng/ml crEPO group, the 20 ng/ml and 10 ng/ml crEPO groups showed similarly low proliferation and enucleation rates (FIG. 7).

There was no morphological difference in the cells even when high concentrations of crEPO were used. The cell proliferation rates of the 80 ng/ml crEPO and 160 ng/ml crEPO groups decreased compared to the 40 ng/ml crEPO group for the first 7 days (FIG. 5). In the 80 ng/ml crEPO group, differentiation progressed similarly to the 40 ng/ml crEPO group, and the largest number of erythrocytes was observed on day 17 day of culture, but cell viability decreased thereafter.

However, higher concentrations of crEPO did not guarantee higher cell proliferation rates on average. Although the proportion of erythrocytes tended to be slightly higher when crEPO was used at higher concentrations, the highest proportion of erythroid lineages compared to the final cell number was observed at crEPO 40 ng/ml (FIG. 7).

Concentrations of canine EPO and human EPO cytokines were compared.

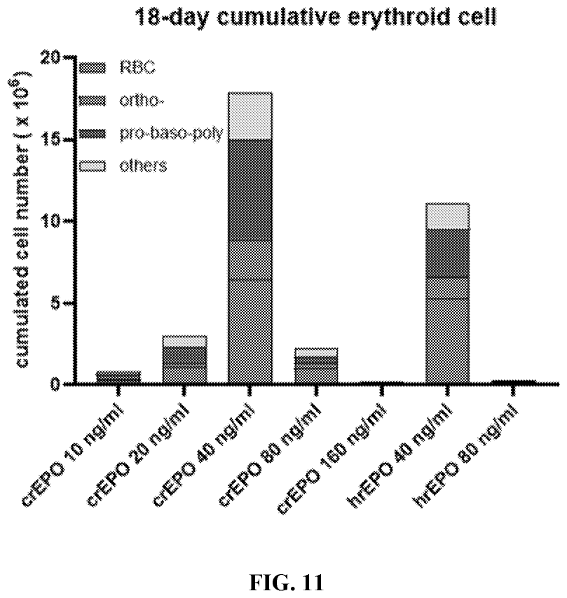

In terms of cell proliferation rate, enucleation, and erythrocyte production, canine EPO 40 ng/ml had a high proliferation rate, and accordingly, the erythrocyte production rate was also high. Human EPO 40 ng/ml had the next best proliferation rate and erythrocyte production, and it was confirmed that erythrocytes were produced even when human EPO was used. The difference in proliferation rate between the canine EPO group and the human EPO group widened from 1.61 to 2.83 times in the period of days 18 to 21 of culture (FIG. 9).

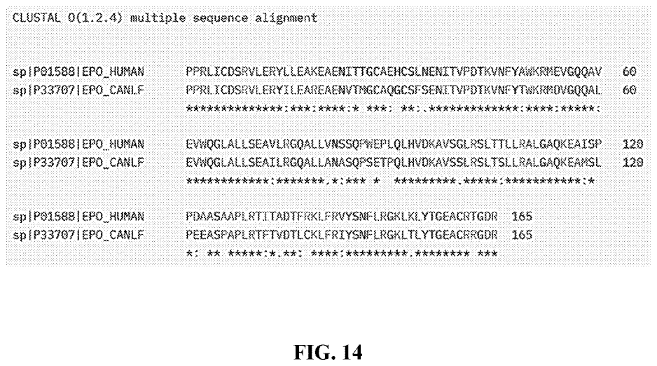

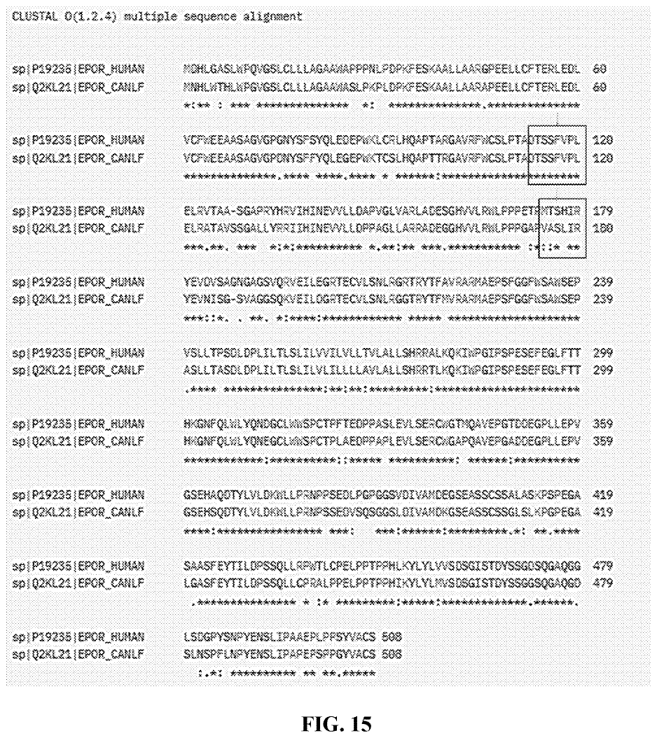

Comparison of the amino acid sequences of human EPO and canine EPO showed an identity of 81.21% and a similarity of 93.93%, and the sequences of human EPOR and canine EPOR were confirmed to have an identity of 82.64% and 91.53% (FIGS. 14 and 15).

### Canine Erythroid Lineage Differentiation and Cell Morphology

The differentiation and maturation of canine erythroblasts may be divided into several distinct stages, which are commonly observed when examining the differentiation process of mammalian cells.

The early stage cells, proerythroblasts, have a high nucleus-to-nucleoplasm ratio, contain ribosomes, and are relatively large cells. As proerythroblasts transform into basophilic erythroblasts, their size decreases, hemoglobin synthesis increases, and the cytoplasm becomes more acidophilic. In cells at this stage, a more condensed chromatin structure appears compared to the previous stage, and the nuclear size begins to decrease.

In polychromatic erythroblasts at the next stage, the cell size decreases further and hemoglobin accumulates more. Furthermore, hemoglobin and residual RNA are mixed, resulting in a pink-tinged cytoplasm in Wright-Giemsa staining. This stage is when hemoglobin synthesis reaches its peak before the cells prepare for enucleation.

Finally, orthochromatic erythroblasts are the smallest of the erythrocyte progenitor cells, and have a dense, condensed nucleus and high hemoglobin content. Generally, the nucleus is skewed toward one side of the cytoplasm, prepares for enucleation and undergoes the process of differentiating into terminal erythrocytes. Thereafter, when enucleation is completed, reticulocytes, which are cells without a nucleus, are formed, eventually becoming red blood cells (RBCs). At this stage, the cell size decreases, and morphological changes in the cell cause a transition into functional erythrocytes, resulting in the cell acquiring the function of efficient oxygen transport.

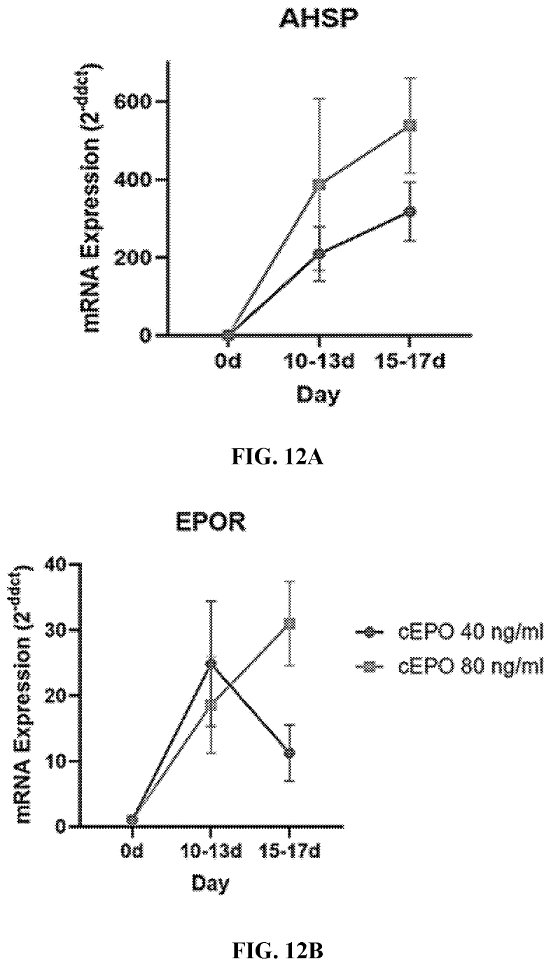

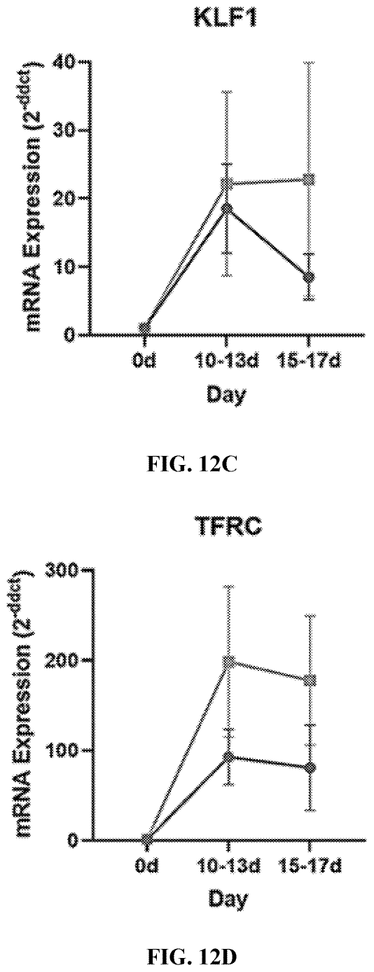

As a result of observing morphological changes according to erythropoiesis while culturing canine PBMCs in vitro, it could be confirmed through Wright-Giemsa staining that canine PBMCs transformed from proerythroblasts to eosinophilic erythroblasts and erythrocytes. When the concentration of EPO cytokine and the degree of differentiation were confirmed, optimal differentiation into erythrocytes according to the cell proliferation rate was achieved at crEPO 40 ng/ml (FIGS. 5 to 11).

Identification of Erythrocyte-Specific mRNA Expression and Erythrocyte Markers

To confirm the mRNA expression of erythrocyte-specific markers, the expression of alpha-hemoglobin stabilizing protein (AHSP) (Reference 9), EPO receptor (EPOR) (Reference 10), Kruppel-like factor 1 (KLF1) (Reference 11), and transferrin receptor 1 (TFRC) (Reference 12) was investigated.

Based on the start date of the experiment, mRNA samples were collected starting from day 7 when proliferation started after administration of crEPO at concentrations of 40 ng/ml and 80 ng/ml. The expression of erythrocyte-specific markers was evaluated at the early stage, days 10 to 13 (FIGS. 2 and 5), when the pellet began to turn red, and at the late stage, days 15 to 17 (FIG. 2), when the enucleated erythrocytes increased and the cell viability was 80%. As a result of the evaluation, it was observed that the mRNA expression levels of AHSP, EPOR, KLF1, and TFRC increased at both concentrations (crEPO 40 ng/ml and 80 ng/ml).

Since there are no previous results analyzing the expression of erythrocyte markers during the erythropoiesis process in dogs, the present inventors compared the mRNA expression results with the mRNA expression patterns of humans and mice.

### AHSP

It is known that the expression of α-hemoglobin stabilizing protein (AHSP) increases when red blood cells are produced from human PBMCs (Reference 13).

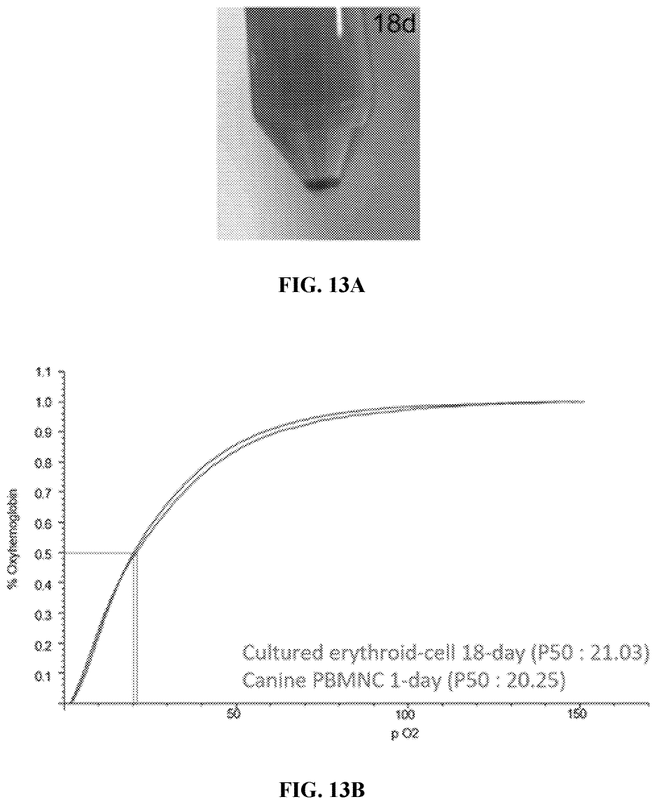

As a result of confirming the expression of α-hemoglobin stabilizing protein (AHSP) in dogs during the culture period, the expression of AHSP mRNA increased as differentiation progressed, similar to the process of erythropoiesis derived from human PBMCs, and this was confirmed at both 40 ng/ml and 80 ng/ml of crEPO (FIG. 12A).

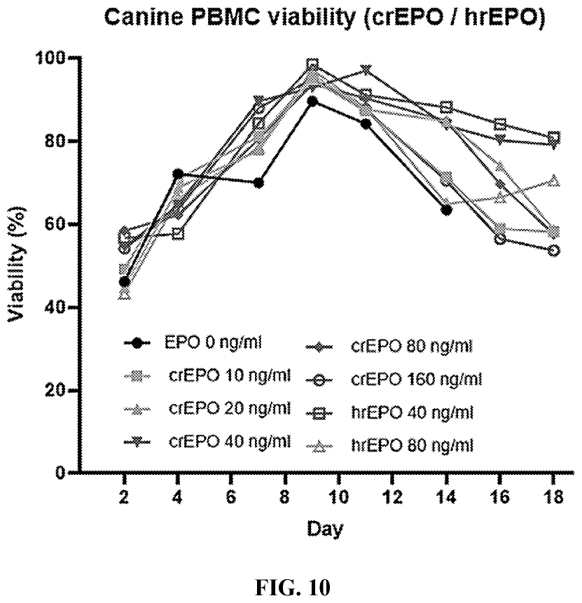

In the case of humans, the highest proportion of polychromatic and orthochromatic erythroblasts was observed between days 8 and 10, which correlated with the peak of AHSP expression. In contrast, during the process of producing red blood cells from canine PBMCs, the proportion of polychromatic and orthochromatic erythroblasts was higher on around day 17 than on around day 10 (FIG. 8). This comparison suggests that the expression of AHSP increases with the differentiation rate in dogs, similar to humans.

### EPOR

For EPOR expression, mRNA expression has been reported to increase during the transition from burst-forming unit-erythroid (BFU-E) to colony-forming unit-erythroid (CFU-E) in humans and mice (Reference 14). In dogs, it is known that EPOR expression is upregulated in chronic hypoxic pulmonary disease, which may induce erythrocytosis as a compensatory mechanism to improve oxygen supply (Reference 15). Further, EPOR is known to gradually decrease during erythroblast maturation in a mouse model (Reference 16).

Although there have been no previous studies on EPOR in canine erythropoiesis, the present inventors confirmed that EPOR expression was reduced at a concentration of 40 ng/ml crEPO in mature erythroblasts (FIG. 12B).

### KLF1 and TFRC

KLF1 plays a critical role in determining the lineage of early megakaryocyte-erythroid progenitors (MEPs), and is expressed in the erythroid lineage to suppress the megakaryocyte lineage. In the present invention, KLF1 expression increased as erythrocytes proliferated, and then relatively decreased as they matured (FIG. 12C).

TFRC is present in large quantities in the cell membrane, and disappears in mature erythrocytes after the nucleus is removed (Reference 17). It is also regulated in relation to iron uptake (Reference 16), and is expressed not only in erythrocytes but also in other hematopoietic lineages (Reference 18). In the present invention, it was confirmed that mRNA expression increased in canine erythropoiesis (FIG. 12D), and although TFRC is not an erythrocyte-specific marker, it showed enhanced expression that may perform a function related to iron uptake.

However, as mRNA expression decreased as mixed hematopoietic lineage cells and erythrocytes underwent enucleation, it was difficult to identify significant trends in mRNA expression according to culture date and erythrocyte markers. However, mRNA expression was slightly higher in the crEPO 80 ng/ml group than in the 40 ng/ml group, but it was not statistically significant.

### Hemoglobin Detection and Oxygen Equilibrium Analysis

Hemoglobin (Hb) α expression was confirmed by Western blot and general PCR.

Two 17-day-old canine donor samples were compared, and the protein expression of Hb α was measured in comparison with GAPDH. As a result of measurement, it can be seen that Hb α was expressed (FIG. 12C).

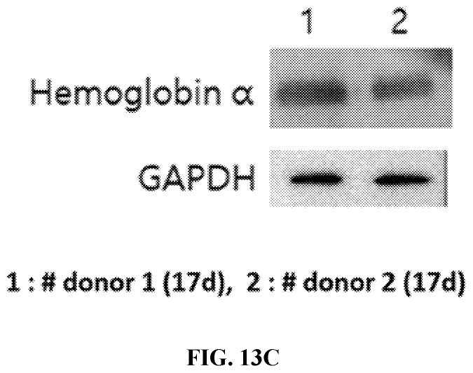

Further, the oxygen equilibrium curves of canine erythrocytes (day 1) and canine PBMCs (crEPO 40 ng/ml) cultured in vitro for 18 days were measured using a Hemox-analyzer. As a result, it could be seen that the oxygen-Hb binding and dissociation curves of canine PBMCs cultured in vitro and canine erythrocytes on day 1 were similar. The P50 values were 21.03 and 20.25, respectively (FIG. 13B). These results indicate that canine PBMC erythrocytes cultured in vitro can produce mature hemoglobin.

### CONCLUSION

Despite the increasing number of companion dogs, virtually no research has been conducted on artificial blood related to dogs, and the exploration of differentiation processes is also minimal. The present inventors successfully produced canine erythrocytes differentiated in vitro for the first time, investigated the characteristics of cell proliferation and differentiation, and evaluated the functional characteristics of cultured erythrocytes.

In early experiments, attempts were made to isolate canine CD34+ cells (References 19 and 20) from canine PBMCs, similar to human cells, and differentiate the canine CD34+cells into specific erythroid lineages. However, CD34+cell isolation was not successful (data not shown), and proliferation was also minimal during a 7-day culture period. In addition, although current commercially available reagents are available for canine use, there is a problem in that the reagents are less efficient than the human reagent. CD34+, a cell marker mainly used to classify erythroid lineages in human PBMCs, did not work effectively in canine cells, and there were no appropriate markers (CD71, CD235a) capable of determining maturation status.

Despite the difficulty in differentiating canine erythrocytes in vitro, the present inventors directly induced erythrocyte differentiation of canine PBMCs by controlling cytokine concentrations, and as a result, were also able to produce erythrocytes in vitro in dogs.

When comparing the proliferation and differentiation rates of erythroid lineages according to the concentration of canine EPO cytokine, proliferation was low at low concentrations below crEPO 40 ng/ml, the expression rates of other hematopoietic lineage cells increased, and erythrocytes were produced. In contrast, at concentrations above crEPO 40 ng/ml, erythrocytes were well produced and mRNA expression markers were also well expressed, but the optimal concentration was set to crEPO 40 ng/ml because the proliferation rate and enucleation rate did not significantly differ between crEPO 40 ng/ml and higher concentrations. In a comparative experiment with human EPO, the concentration of human EPO 40 ng/ml (3 to 15 times higher than the utilized activity value of human EPO) showed graphs indicating high proliferation and differentiation, second only to crEPO 40 ng/ml, but when comparing the relative erythrocyte production amounts, it did not exceed the proliferation rate of crEPO 40 ng/ml (2.83 times).

As a result of analyzing the P50 curve of oxygen binding-dissociation for functional analysis of erythrocytes, similar curves were found between canine erythrocytes cultured in vitro and canine erythrocytes derived from PB. This confirmed that canine erythrocytes cultured in vitro function well compared to peripheral blood erythrocytes.

### REFERENCES

1) DOI: 10.1182/blood-2004-03-1002
2) doi: 10.1182/blood-2011-06-36203
3) Immune phenotype of canine hematopoietic progenitor cells, https://doi. org/10.1111/j.1399-0039.1997.tb02901.x
4) Comparison of Phenotypic and Functional Characteristics Between Canine Non-B, Non-T Natural Killer Lymphocytes and CD3+CD5dimCD21− Cytotoxic Large Granular Lymphocytes, doi:10.3389/fimmu.2018.00841
5) Red blood cells morphology and morphometry in adult, senior, and geriatricians dogs by optical and scanning electron microscopy, https://doi. org/10.3389/fvets.2022.9984382
6) ZooMics: comparative metabolomics of red blood cells from dogs, cows, horses and donkeys during refrigerated storage for up to 42 days, doi: 10.2450/2022.0118-22
7) England, S., McGrath, K., Frame, J., & Palis, J. (2011). Immature erythroblasts with extensive ex vivo self-renewal capacity emerge from the early mammalian fetus. Blood, 117(9), 2708-2717. https://doi.org/10.1182/blood-2010-07-299743
8) Hedayanti, N. (2023). Review of physiological aspects of erythropoiesis: a narrative literature review. sjim, 1(1), 19-25. https://doi.org/10.59345/sjim.v1i 1.20
9) Alpha-hemoglobin stabilizing protein is a sensitive and specific marker of erythroid precursors, doi:10.1097/PAS.0b013e31825fa501
10) Erythropoietin regulation of red blood cell production: from bench to bedside and back First https://doi.org/10.12688/f1000research.26648.1
11) A novel mutation in the erythroid transcription factor KLF1 is likely responsible for ameliorating β-thalassemia major, DOI: 10.1002/humu.23817
12) Using microarray analysis to identify genes and pathways that regulate fetal hemoglobin levels, DOI: 10.1111/ahg.12346
13) Expression of α-hemoglobin stabilizing protein gene during human erythropoiesis, doi: 10.1016/j. exphem.2003.11.002
14) Core erythropoietin receptor signals for late erythroblast development, DOI: 10.1182/blood-2005-02-0684
15) Polycythemia in dogs with chronic hypoxic pulmonary disease, 10.1111/jvim.16466
16) A mouse model for visualization and conditional mutations in the erythroid lineage, https://doi. org/10.1182/blood-2003-05-1442
17) fate of the transferrin receptor during maturation of sheep reticulocytes in vitro: selective externalization of the receptor, DOI: 10.1016/0092-8674(83)90040-5
18) CD71 (Transferrin Receptor): An Effective Marker for Erythroid Precursors in Bone Marrow Biopsy Specimens, DOI: 10.1309/AJCPCRK3MOAOJ6AT
19) Efficient enucleation of erythroblasts differentiated in vitro from hematopoietic stem and progenitor cells, doi:10.1038/nbt1245
20) Application of mutant JAK2V617F for in vitro generation of red blood cells, doi:10.1111/trf.13431
21) A Method to Isolate CD34+ Mononuclear Cells from Canine Peripheral Blood, A Method to Isolate CD34+ Mononuclear Cells from Canine Peripheral Blood
22) Isolation and characterization of pediatric canine bone marrow CD34+ cells, doi:10.1016/j.vetimm.2004.03.009.
23) Hospital-acquired Anemia in Critically Ill Dogs and Cats: A Multi-Institutional Study, DOI: 10.1111/jvim.13650

1. A method for producing canine erythrocytes in vitro, the method comprising: obtaining hematopoietic stem cells or erythroid progenitor cells from canine-derived cells; and
culturing the obtained hematopoietic stem cells or erythroid progenitor cells in a medium containing erythropoietin (EPO).

2. The method of claim 1, wherein the canine-derived cells are blood-derived cells, bone marrow-derived cells, cord blood-derived cells, or stem cells.

3. The method of claim 1, wherein the hematopoietic stem cells are mononuclear cells.

4. The method of claim 1, wherein the erythropoietin is included in the medium at a concentration of 20 to 80 ng/ml.

5. The method of claim 4, wherein the erythropoietin is included in the medium at a concentration of 30 to 50 ng/ml.

6. The method of claim 1, wherein the erythropoietin is canine erythropoietin.

7. The method of claim 1, wherein the erythropoietin is human erythropoietin.

8. The method of claim 1, wherein the medium further comprises transferrin, insulin, penicillin/streptomycin, and amino acids.

9. The method of claim 1, wherein the culturing step is performed for 3 days to 30 days.

10. The method of claim 6, wherein the canine erythropoietin (EPO) is included in the medium at a concentration of 5 to 80 ng/ml.

11. The method of claim 1, wherein the medium further comprises stem cell factor (SCF) and interleukin-3.

12. The method of claim 11, wherein the stem cell factor (SCF) is included in the medium at a concentration of 10 to 100 ng/ml.

13. The method of claim 11, wherein the interleukin-3 is included in the medium at a concentration of 1 to 10 ng/ml.

14. Erythrocytes produced according to the method of claim 1.
